(19) United States
(12) Patent Application Publication
(10) Pub. No.: US 2020/0276442 A1
(43) Pub. Date: Sep. 3, 2020

(54) WAVE-FORM METHOD FOR ACCELERATING BLOOD FLOW

(71) Applicant: VasoActiv Biomedical Technologies LLC, Tulsa, OK (US)

(72) Inventor: John M. Owen, Tulsa, OK (US)

(21) Appl. No.: 16/567,744

(22) Filed: Sep. 11, 2019

Related U.S. Application Data
(60) Provisional application No. 62/811,990, filed on Feb. 28, 2019.

(51) Int. Cl. A61N 1/36; A61N 1/04

(52) U.S. Cl. CPC A61N 1/36014; A61N 1/0492; A61N 1/0456; A61N 1/0452

(57) ABSTRACT

A method and protocol for application of neuromuscular electrical stimulation (NMES) to the skeletal muscles using a plurality of treatment pads (electrodes) on all extremities, activated sequentially, with overlapping timing, distal to proximal, with the impulses released in a similar, following pattern to enhance the refill cycle, resulting in wave-form acceleration of blood flow in cardiovascular circulation.

### CROSS-REFERENCE TO RELATED CASES

This application claims the benefit of U.S. provisional patent application Ser. No. 62/811,990, filed on Feb. 28, 2019, and incorporates such provisional application by reference into this disclosure as if fully set out at this point.

### FIELD OF THE INVENTION

This disclosure related to improving blood flow in general and, more specifically, to a timing protocol of neuromuscular stimulation as a means to accelerate blood flow.

### BACKGROUND OF THE INVENTION

Blood is a non-Newtonian fluid, that is, a fluid whose viscosity is variable based on applied stress or force. In the human body, the physical behavior of blood depends on the forces acting on it from second to second.

Poor circulation is a serious condition that can contribute to a number of health problems. Symptoms of poor circulation can include pain when walking, chest pain during exertion, high blood pressure, infections in the feet from decreased blood flow or trouble seeing. Poor circulation can result in kidney damage resulting in fatigue, fluid retention and protein in the urine in the early stages, and can cause kidneys to fail completely, requiring dialysis to remove waste products from blood or a kidney transplant. Poor circulation leads to skin breakdown and infection, especially in the feet. Poor circulation in the bedridden leads to decubitis ulcers (also called bed sores or pressure ulcers), a painful and potentially fatal condition.

People with diabetes have a much higher risk of foot or leg amputation due to the increased risk of infection from decreased blood flow through damaged vessels. Poor circulation is also a major contributor to neuropathy, which is extremely painful and often leads to amputation. Smokers with diabetes have the greatest risk of amputation because smoking also constricts blood vessels.

Poor circulation contributes to increased coagulation and can be dangerous, especially when not properly identified and treated. People with hypercoagulable states have an increased risk for blood clots developing in the arteries and veins. Blood clots in the venous system can travel through the bloodstream and cause deep vein thrombosis or a pulmonary embolism. Blood clots in the arteries can increase the risk for stroke, heart attack, severe leg pain, difficulty walking, or even the loss of a limb.

Circulatory collapse is a marker for extreme sepsis and septic shock, which leads to organ failure and death. Sepsis has a mortality rate of 15 to 30 percent, with late stage sepsis reaching a mortality rate of 50%.

Clearly, there is a need for a method to accelerate and otherwise improve cardiovascular blood flow. Methods of the present disclosure accomplish this and other goals by, among other things, using a new and improved timing protocol that is more effective at producing blood movement.

### SUMMARY OF THE INVENTION

The invention of the present disclosure, in one aspect thereof, comprises a method including applying a first plurality of pairs of electric treatment pads to a first limb of a patient from a distal to a proximal location on the first limb, and providing an electrical neuromuscular stimulation to the first plurality of pairs of treatment pads according to a wave-form. The wave-form is applied in a sequential and overlapping manner to the first plurality of pairs of treatment pads such that the electrical neuromuscular stimulation progresses from the distal to the proximal location on the first limb. The wave-form activates a first most distal pair of pads of the first plurality of treatment pads and thereafter activates a second most distal pair of pads of the first plurality of treatment pads while keeping the first most distal pair of pads of the first plurality of treatment pads activated. Finally, the wave-form deactivates the first most distal pair of pads of the first plurality of treatment pads when a third most distal pair of pads of the first plurality of treatment pads is activated.

The method may also include applying a second plurality of pairs of electric treatment pads to a second limb of the patient from a distal to a proximal location on the second limb, and providing the electrical neuromuscular stimulation to the second plurality of pairs of treatment pads according to the predetermined wave-form. The wave-form is applied in a sequential and overlapping manner to the second plurality of pairs of treatment pads such that the electrical neuromuscular stimulation progresses from the distal to the proximal location on the second limb. The wave-form activates a first most distal pair of pads of the second plurality of treatment pads and thereafter activates a second most distal pair of pads of the second plurality of treatment pads while keeping the first most distal pair of pads of the second plurality of treatment pads activated. As before, the wave-form deactivates the first most distal pair of pads of the second plurality of treatment pads when a third most distal pair of pads of the second plurality of treatment pads is activated.

The method of may further include applying a third plurality of pairs of electric treatment pads to a third limb of the patient from a distal to a proximal location on the third limb, and providing the electrical neuromuscular stimulation to the third plurality of pairs of treatment pads according to the predetermined wave-form. The wave-form is applied in a sequential and overlapping manner to the third plurality of pairs of treatment pads such that the electrical neuromuscular stimulation progresses from the distal to the proximal location on the third limb. The wave-form activates a first, most distal pair of pads of the third plurality of treatment pads and thereafter activates a second most distal pair of pads of the third plurality of treatment pads while keeping the first most distal pair of pads of the third plurality of treatment pads activated. The wave-form deactivates the first most distal pair of pads of the third plurality of treatment pads when a third most distal pair of pads of the third plurality of treatment pads is activated.

The method may further include applying a fourth plurality of pairs of electric treatment pads to a fourth limb of the patient from a distal to a proximal location on the fourth limb, and providing the electrical neuromuscular stimulation to the fourth plurality of pairs of treatment pads according to the predetermined wave-form. The wave-form is applied in a sequential and overlapping manner to the fourth plurality of pairs of treatment pads such that the electrical neuromuscular stimulation progresses from the distal to the proximal location on the fourth limb. The wave-form activates a first most distal pair of pads of the fourth plurality of treatment pads and thereafter activates a second most distal pair of pads of the fourth plurality of treatment pads while keeping the first most distal pair of pads of the fourth plurality of treatment pads activated. Finally, as with the other limbs, the wave-form deactivates the first most distal pair of pads of the fourth plurality of treatment pads when a third most distal pair of pads of the fourth plurality of treatment pads is activated.

In some embodiments, the first limb is an arm. The first limb may also be a leg. In other cases, the first limb and second limb are arms, but the first limb and second limb can be legs. The first limb may be a leg and the second limb an arm. In some cases, the first and second limbs are left and right arms, respectively, and the third and fourth limbs are left and right legs, respectively. In such case, the wave-form may apply to the first and third plurality of treatment pads simultaneously, followed by application of the wave-form to the second and fourth treatment pads simultaneously. In some cases, application of the wave-form to the first and third plurality of treatment pads does not overlap with application of the wave-form to the second and fourth plurality of treatment pads. The electrical neuromuscular stimulation at each pair of the first plurality of pairs of treatment pads may be about 500 ms in duration.

The invention of the present disclosure, in another aspect thereof, comprises a method including applying a first plurality of electrically conductive treatment pads along plurality of locations along a first limb of a patient in need of treatment, the locations along the first limb being from distal to proximal, and providing an electrical wave-form as an application of current to the first plurality of electrically conductive treatment pads. The electrical wave-form is applied in a sequential and overlapping manner to the first plurality of treatment pads such that an electrical neuromuscular stimulation progresses from the distal to proximal locations on the first limb. The electrical wave-form applies the electrical neuromuscular stimulation such that stimulation occurs at two adjacent locations on the first limb, except for a beginning of the wave-form when only a most distal location of the first limb receives stimulation and an end of the wave-form when only a most proximal location on the first limb receives stimulation. The electrical wave-form maintains stimulation at no more than two adjacent locations on the first limb at any time.

The previous method may also include applying a second plurality of electrically conductive treatment pads along plurality of locations along a second limb of the patient in need of treatment, the locations along the first limb being from distal to proximal, and providing the electrical wave-form as an application of current to the second plurality of electrically conductive treatment pads. The electrical wave-form is applied in the sequential and overlapping manner to the second plurality of treatment pads such that an electrical neuromuscular stimulation progresses from the distal to proximal locations on the second limb. The electrical wave-form applies the electrical neuromuscular stimulation such that stimulation occurs at two adjacent locations on the second limb, except for a beginning of the wave-form when only a most distal location of the second limb receives stimulation and an end of the wave-form when only a most proximal location on the second limb receives stimulation. Again, the electrical wave-form maintains stimulation at no more than two adjacent locations on the second limb at any time.

In such cases, the first limb may be an arm of the patient and the second limb a leg of the patient. The electrical wave-form may be applied to the plurality of electrically conductive treatment pads on the first limb and to the plurality of electrically conductive treatment pads on the second limb simultaneously.

The invention of the present disclosure, in another aspect thereof, comprises a device including an electrical wave-form generator, a first plurality of pairs of electric treatment pads adapted to attach to a first limb of a patient from a distal to a proximal location on the first limb, each of the pairs of electrical treatment pads of the first plurality of pairs of electrical treatment pads, when active, providing electrical neuromuscular stimulation at their respective location to the first limb of the patient, and leads electrically connecting the first plurality of pairs of electric treatment pads to the wave-form generator. The electrical wave-form generator provides a stimulation wave-form to the first plurality of pairs of electrical treatment pads and the stimulation wave-form activates the first plurality of pairs of treatment pads in a sequential and overlapping manner such that the electrical neuromuscular stimulation progresses from the distal to the proximal location on the first limb. The stimulation wave-form also activates a first most distal pair of pads of the first plurality of treatment pads and thereafter activates a second most distal pair of pads of the first plurality of treatment pads while keeping the first most distal pair of pads of the first plurality of treatment pads activated. The stimulation wave-form deactivates the first most distal pair of pads of the first plurality of treatment pads when a third most distal pair of pads of the first plurality of treatment pads is activated.

The device may also include a second plurality of pairs of electric treatment pads adapted to attach to a second limb of a patient from a distal to a proximal location on the second limb, each of the pairs of electrical treatment pads of the second plurality of pairs of electrical treatment pads, when active, providing electrical neuromuscular stimulation at their respective locations to the second limb of the patient, and leads electrically connecting the second plurality of pairs of electric treatment pads to the wave-form generator. The electrical wave-form generator provides the stimulation wave-form to the second plurality of pairs of electrical treatment pads. The stimulation wave-form activates the second plurality of pairs of treatment pads in a sequential and overlapping manner such that the electrical neuromuscular stimulation progresses from the distal to the proximal location on the second limb. The stimulation wave-form activates a first most distal pair of pads of the second plurality of treatment pads and thereafter activates a second most distal pair of pads of the second plurality of treatment pads while keeping the first most distal pair of pads of the first plurality of treatment pads activated. The stimulation wave-form deactivates the first most distal pair of pads of the second plurality of treatment pads when a third most distal pair of pads of the second plurality of treatment pads is activated.

In some embodiments, the wave-form generator provides the stimulation wave-form to the first and second pluralities of treatment pads simultaneously.

### BRIEF DESCRIPTION OF THE DRAWINGS

FIG. 1 is a schematic drawing showing a blood vessel and plurality treatment pads prior to activation according to aspects of the present disclosure.

FIG. 2 is a schematic drawing showing the blood vessel and pads of FIG. 1 at initiation of a treatment sequence.

FIG. 3 is a schematic drawing showing the blood vessel and pads of FIG. 1 as treatment continues from FIG. 2.

FIG. 4 is a schematic drawing showing the blood vessel and pads of FIG. 1 as treatment continues from FIG. 3.

FIG. 5 is a schematic drawing showing the blood vessel and pads of FIG. 1 as treatment continues from FIG. 4.

FIG. 6 is a schematic drawing showing the blood vessel and pads of FIG. 1 as treatment continues from FIG. 5.

FIG. 7 is a schematic drawing showing the blood vessel and pads of FIG. 1 as treatment continues from FIG. 6.

FIG. 8 is a drawing of a wave-form stimulation device for providing treatments according to aspects of the present disclosure.

FIG. 9 is a diagram of a patient showing exemplary placement of treatment pads.

FIG. 10 is a chart of exemplary interrelationship of effects and therapeutic results from accelerated wave-form blood movement according to methods of the present disclosure.

FIG. 11 is a simplified diagram of a human patient leg further illustrating possible treatment pad locations.

### DETAILED DESCRIPTION OF THE PREFERRED EMBODIMENTS

Electrical stimulation has been in use since Galvani's experiments in the 1700's. Common uses include: relaxation of muscle spasms, prevention or retardation of disuse atrophy, increasing local blood circulation, muscle re-education, immediate post-surgical stimulation of calf muscles to prevent venous thrombosis, and maintaining or increasing range of motion. Previous applications of neuromuscular electrical stimulation (LAMES) for the purpose of moving blood have not been designed for maximizing blood flow velocity as the current disclosure provides.

The present disclosure provides various embodiments of new and improved methods and devices for accelerating blood flow/cardiovascular circulation by use of a new protocol for pulse timing of neuromuscular stimulation. In some embodiments, the stimulation protocol comprises a series of impulses timed in sequential, overlapping order from distal to proximal, with the impulses released in a similar, following pattern to enhance the refill cycle. In other words, the stimulation is sequentially ceased, distal to proximal in a like manner and timing to the stimulation initiation sequence. This robust wave-form blood flow, which is similar in organization to the peristaltic wave-form movement of food through the esophagus and intestines, is an improvement over existing methods and results in an improved result.

Embodiments of the wave-form blood flow protocol create both a greater volume of blood moved and a higher velocity of movement. The former is helpful for delivering elevated quantities of oxygen, medicine and nutrients to tissue, the latter is beneficial in elevating endothelial shear stress resulting in stimulation of the body's autocrine and paracrine processes, bringing about significant and beneficial changes in the patient's blood chemistry.

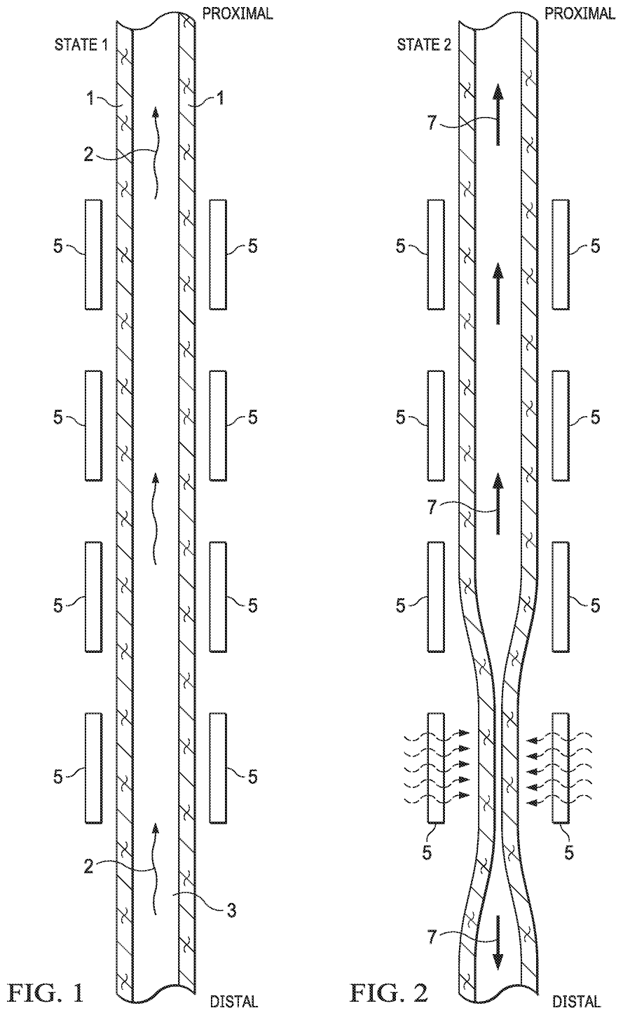

Referring now to FIG. 1, a stylized/schematic rendering of a blood vessel 4 is shown. For purposes of illustration the blood vessel 4 is shown in the absence of muscle, bone, skin, and the like. The blood vessel 4 may be any blood vessel in the body but with respect to particular embodiments of the present disclosure, the blood vessel 4 is a large vein in the foot, leg, hand, or arm, such as a tibial or saphenous vein. It is known that certain large veins within the human body have one-way valves as a part of the anatomy. In some respects, in a healthy individual, such veins can serve to eliminate or reduce “retrograde” flow of blood through the veins which would be in a distal direction, as blood normally travels in an artery. It should be appreciated that methods of the present disclosure provide therapeutic effect with respect to action upon particular veins whether such veins are those having internal valves or not. Hence, such valves are not illustrated in FIGS. 1-7.

Anatomically, the interior layer of the vein 4 is the endothelial layer 1. This is the innermost layer of a vein that is in actual contact with blood flow 2 and defines the inner flexible lumen 3 of the vein 4. The influence of the endothelium is far reaching and is more than simply a conduit for blood. It is the largest organ in the body and would be equivalent in size to approximately six tennis courts if spread out. It exerts control over an array of mechanisms which serve to maintain vascular tone and blood fluidity by maintaining vascular smooth muscle tone, regulating angiogenesis and cell proliferation, mediating inflammatory and immune responses, regulating vascular permeability, regulating thrombolysis, regulating leukocyte adhesion, regulating platelet adhesion and aggregation, and regulating lipid oxidation, among other actions and effects.

The endothelium exerts such control through endocrine, paracrine and autocrine processes wherein the endothelial cells secrete vasoactive substances such as hormones, genes, proteins, transcription factors and others, resulting in the regulatory actions listed above. This group of events is generally known as, “endothelial mechanotransduction.” Mechanotransduction refers to the processes through which cells sense and respond to mechanical stimuli by converting them to biochemical signals that elicit specific cellular responses.

Endothelial mechanotransduction happens in response to blood flow and laminar shear stress, induced from the mechanical forces caused by the rubbing of blood cells on the endothelium (the lining of blood vessels). When people are young, the normal physiologic levels of blood flow and shear stress keep blood vessels (and the whole cardiovascular system) healthy. Later in life, people make diet and lifestyle choices that can lower blood flow, clog the blood vessels with fatty deposits and impair the regulatory processes necessary for vascular health. The endothelium can then become dysfunctional contributing to atherosclerosis (hardening of the arteries), diabetes, hypertension (high blood pressure), delayed wound healing, vasculitis, congestive heart failure, critical limb ischemia, neuropathy and more.

Methods of the present disclosure positively affect the endothelium by improving vascular return of blood from the extremities of a patient. However, other benefits of aiding return of blood flow not directly related to the endothelium per se may also be observed. Thus, the present disclosure and the effects of the methods herein are not strictly limited to those that rely upon endothelial effects. Further, there may exist in the prior art certain devices and methods that can be observed to improve return blood flow and possibly endothelial function. However, in various embodiments, the present disclosure presents an improved “wave form” that can be applied to a plurality of treatment pads placed on one or more extremities that stimulate blood vessels and the endothelial layer by utilizing the patient's own skeletal muscle as a “pump”. It has been known that such a pumping action is affected by normal movement of a person, particularly in walking, but one who is immobile or otherwise unable to tolerate walking, for example, does not derive the full benefit of this anatomical pump.

In accordance with embodiments of the present disclosure, electrical stimulation pads 5 may be applied in pairs on opposite sides of a patient's limb. Electrical stimulation applied to the skin can result in contraction of muscle tissue surrounding the vein and provide a pumping action according to the wave-forms and methods herein. In reality, many blood vessels may run within any limb or extremity such that one or more veins receive the benefit of the stimulation described herein.

Distal and proximal ends are labelled in FIG. 1. In the case of application of the pads 5 to a patient's leg, the distal end represents the feet and the proximal end represents the upper thigh, for example. As shown in FIG. 1, the endothelium 1 and surrounding muscle are relaxed, blood flow 2 is weak through the lumen 3. Four pairs of treatment pads 5 are distributed along the limb from distal to proximal. Treatment pads 5 (also known as electrodes) may be applied to the patient either by self-adhesive means or straps or in a garment. In some embodiments, the pads 5 are applied in pairs, opposed 180° on the feet, calves, lower thighs, and upper thighs; the hands, forearms, biceps, and shoulders, although other placements are acceptable and may achieve the desired results. In some embodiments, in order to achieve the desired therapeutic threshold of wave-form blood movement, a minimum of 4 pairs of pads on each extremity must be used.

Referring now to FIG. 2, a schematic drawing showing the blood vessel 4 and pads 5 of FIG. 1 at initiation of a treatment sequence. Here, the most distal pair of pads 5 has been activated by application of current resulting in squeezing or closing of a portion of the lumen 3 by surrounding skeletal muscle. For purposes of the present disclosure, it is understood that voltage is also applied, and the particular relationship between applied voltage and applied current may rest upon a number of factors including the impedance of the pads 5 and the patient's body. In some embodiments, voltage may be applied to one pad out of a pair while the opposite pad acts as ground, or is supplied with a negative voltage thereby increasing current flow or voltage differential even further (within safe limits) while limiting the amount of voltage (positive or negative) applied to any single pad. In any event, blood flow 7 may be (or occur, or move) both proximal and distal at this stage, particularly if the vein 4 is a vein without anatomical valves or if the valves are weak or otherwise ineffective.

Referring now to FIG. 3 a schematic drawing showing the blood vessel 4 and pads 5 of FIG. 1 as treatment continues from FIG. 2 is shown. Here an overlapping, sequential protocol wave-form, state 3, of the present disclosure can being to be seen. The second most distal pair of pads 5 receive current causing muscle contractions which squeeze the blood vessel 4, closing the lumen 3 and forcing blood flow 7 from the area. None of the blood flow 7 is forced distally since the first pair of pads is still receiving current. Additionally, the blood flow 7 may be more forceful that that experienced at rest. Particularly if the patient is in μl health or non-ambulatory. The blood flow 7 is substantial enough to induce shear stress and activation of the endothelial layer as discussed herein.

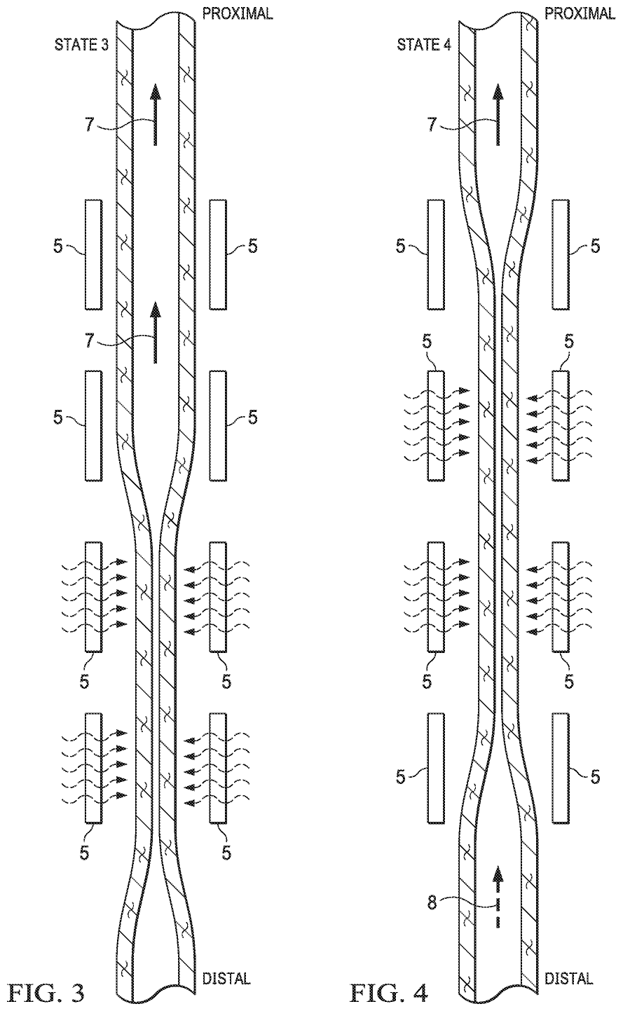

Referring now to FIG. 4, a schematic drawing showing the blood vessel and pads of FIG. 1 as treatment continues from FIG. 3 is shown. FIG. 4 shows the showing of the overlapping, sequential protocol, state 4. Here, the third most distal pair of pads 5 receives current causing muscle contractions which squeeze the blood vessel 4, closing the lumen 3 and forcing blood 7 further from the area (in the proximal direction). None, or at least very little, of the blood is forced distally since the second most distal pair of pads 5 (adjacent in the distal direction) is still receiving current. Current to the first pair of pads activated is terminated causing the blood vessel 4 to be allowed to expand and begin drawing refill blood 8 into the lumen 3.

Referring now to FIG. 5, a schematic drawing showing the blood vessel 4 and pads 5 of FIG. 1 as treatment continues from FIG. 4 is shown. FIG. 5 shows the overlapping, sequential protocol, state 5. The fourth most distal pair of pads 5 receive current causing muscle contractions which squeeze the blood vessel 4, closing the lumen 3 and forcing blood 7 from the area further proximally. Again, little or none of the blood 7 is forced distally since the third most distal pair of pads is still receiving current. Current to the second pair of pads (second most distal and also second activated) is terminated after activation of most proximal pair of pads the allowing the blood vessel 4 to expand even further toward the proximal direction and continue to draw refill blood 8 deeper into the lumen 3.

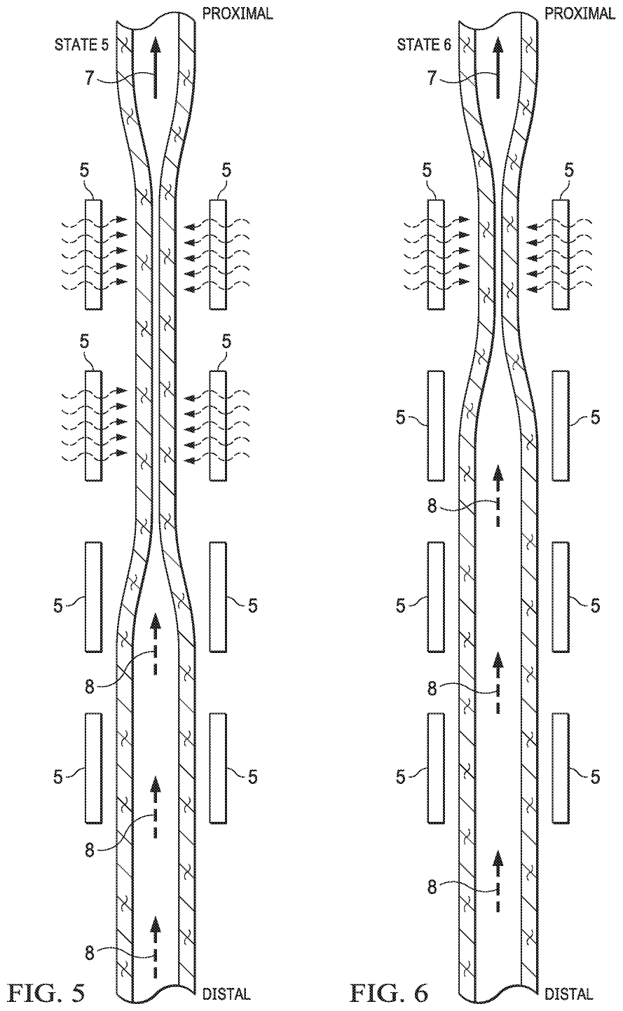

Referring now to FIG. 6, a schematic drawing showing the blood vessel 4 and pads 5 of FIG. 1 as treatment continues from FIG. 5 is shown. FIG. 6 shows the overlapping, sequential protocol, state 6. Current to the third pair of pads 5 (third distally and also third activated) is terminated allowing the blood vessel 4 to expand and continue drawing refill blood 8 deeper into the lumen 3.

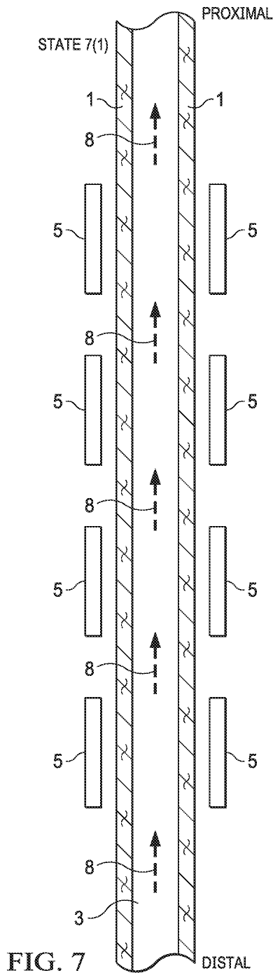

Referring now to FIG. 7, is a schematic drawing showing the blood vessel 4 and pads 5 of FIG. 1 as treatment continues from FIG. 6. Current to the fourth pair of pads 5 (most proximal) is terminated allowing the blood vessel 4 to re-expand along the entire length of the treatment draw refill blood 8 deeper into the lumen 3. This illustrate state, following application of a full wave form through the full set of pads 5 is substantially similar to state 1, FIG. 1. However, blood flow 8 is moving with more force than before (e.g., more forcefully than blood flow 2, FIG. 1). This is the major result of overlapping, sequential timing and the plurality of treatment pads according to embodiments of the present disclosure.

Although the sequence of FIGS. 1-7 illustrates a treatment mode employing four pairs of pads 5, it should be understood that more or fewer pairs of pads 5 may be employed. However, the overlapping aspects of the treatment wave form method would require at least two pairs of pads. Additionally, four pairs as shown provide sufficient stimulation of muscles along a limb so as to enhance proximal blood flow from an extremity to the patient's heart. This is called venous return, and results, according to the Frank-Starling law in higher preload and stroke volume. The wave-form method thus raises cardiac output which in many disease states (such as sepsis) is highly desirable (see, e.g., FIG. 9). If, for some reason, further stimulation points are desired, more than four pairs of pads 5 may be provided and it may be possible to activate a second wave-form before the first has completed (if sufficient distance has been provided between them that there is sufficient return blood flow 8 to be “pushed” by a second wave-form).

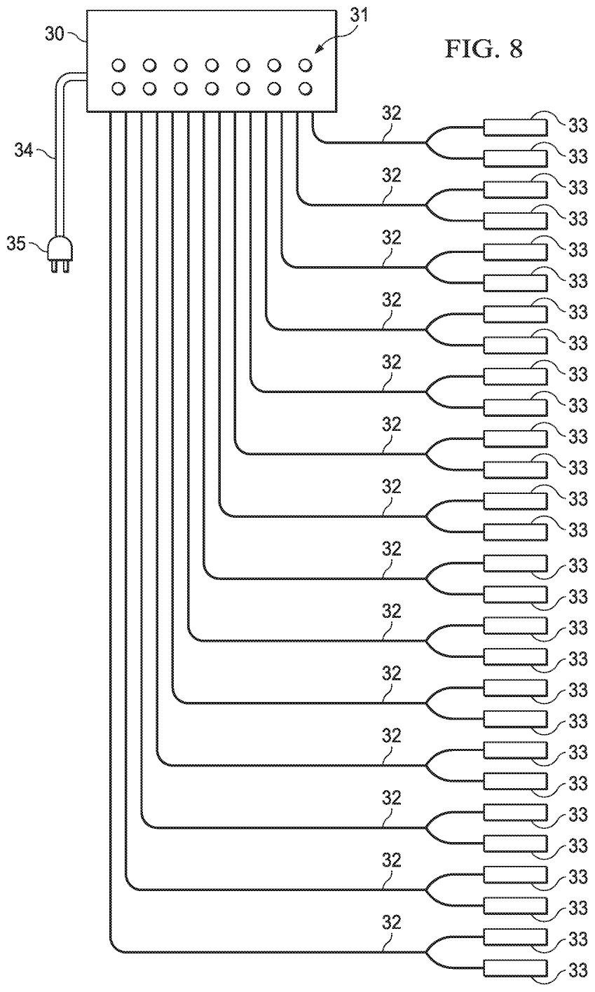

Referring now to FIG. 8, a drawing of a wave-form stimulation device 30 for providing treatments according to aspects of the present disclosure is shown. The wave-form stimulation device may be, in effect, a signal generator. Thus, it may include all necessary hardware and controls as are known in the art to safely apply various electrical signals, currents and voltages that are therapeutic yet safe for the human body. The wave-form stimulation device 30 comprises a plurality of leads 32. Each lead 32 attaches to a pair of treatment pads 33 (corresponding to the treatment pads 5 of FIGS. 1-7). An electrical cord 34 and plug 35 for alternating current (AC) input from a wall socket is provided. The treatment device 30 contains the necessarily internal hardware to convert the AC power to direct current (DC) for safe application to the patient via the leads 32 and pads 33. A number of controls 31 including necessary knobs, dials, levers, switches, and the like are provided to enable the operating therapist to control current/voltage applied within safe but therapeutically effective parameters.

In some embodiments, the leads 32 may be divided into groups of four, such that four pairs of pads may be applied to an extremity or limb of a patient. The number of leads 32 may vary. In some embodiments, at least 8 pairs of leads are provided such that both arms or both legs of a patient may have at least four pairs of pads applied in sequence. In another embodiment, 16 pairs of leads are provided such that both arms and both legs may be provided with four pairs of leads and all extremities be subject to the therapeutic application of the electrical wave-forms discussed herein.

Referring now to FIG. 9 a diagram of a patient 900 showing exemplary placement of treatment pads is shown. Here treatment pads 21 each represent a pair of pads (located, for example, on opposite sides of the limb) and correspond to pads 33 (FIG. 8) and pads 5 (FIGS. 1-7). In some embodiments, treatment may be applied to each limb one at a time. For example, each arm separately and then each leg separately. In another embodiment, treatment of an arm may be followed by treatment of a leg, then the opposite arm and opposite leg. In further embodiments, two limbs may receive treatment simultaneously. This could be, for example, both arms or both legs, but additional benefit may be derived from treatment of one arm and the opposite leg simultaneously and then the opposite of each (for example, left arm with right leg, then right arm with left leg). Such opposite side arm and leg treatment simultaneously may mimic what would be more natural venous blood return in a healthy individual who is walking or running. Of course, it can be envisioned to treat all four limbs simultaneously, but adequate therapeutic results appear to be achieved without such relatively large amount of current applied to the body. Moreover, there appears to be some potential benefit to not treating both arms or both legs at once as this minimizes the potential for current passing through the torso itself. Even if this is not a harmful amount of current, it does not provide the level of therapeutic benefit as treatment applied to and within the extremities.

According to some embodiments, amplitude is adjustable by the operator starting at a level which elicits no response and is gradually increased in amplitude until the patient experiences discomfort, then reduced backed to a comfort level.

In one embodiment, current is sent to the treatment pads attached to one or more limbs in the following manner:

1. the most distal pads receive current, then

2. ˜250 ms later, the second most distal pads receive current, then

3. ˜250 ms later, the third most distal receive current while, simultaneously, the current to the first pads is terminated.

4. ˜250 ms later, the fourth most distal receive current while simultaneously the current to the second pads is terminated.

5. ˜250 ms later current to the third most distal pads is terminated.

6. ˜250 ms later current to the fourth most distal pads is terminated.

As the muscles in the first pads are activated, the muscles in that area contract, squeezing the veins and forcing blood toward the heart. Of course, some blood is also forced away from the heart depending on the location and the presence of venous valves, which can prevent retrograde flow. As the second pads are activated, blood is forced toward the heart and cannot flow away from the heart because the first pads are still activated, and the muscles are preventing the flow of the blood in that direction.

When the muscles relax after termination of each stimulus, the vessel walls return to their uncompressed state thus enlarging the luminal space, an action which draws blood into the vessel. This action is basically “priming the pump” for the next cycle of contractions. A complete cycle of contractions takes a little more than a second, according to the present embodiment. In some of the embodiments, this series of contractions is carried out simultaneously on one leg and the contralateral arm, then switched to the opposite leg-arm combination. In a continuing application of this protocol, this series of muscle contractions will mimic the metabolic demands of a brisk walk and the patient will receive metabolic benefits similar to taking a brisk walk. If administered for 30 to 60 days, the therapy should bring the vasculature closer to homeostasis so that the higher level of blood flow will be sustained well beyond the treatment time.

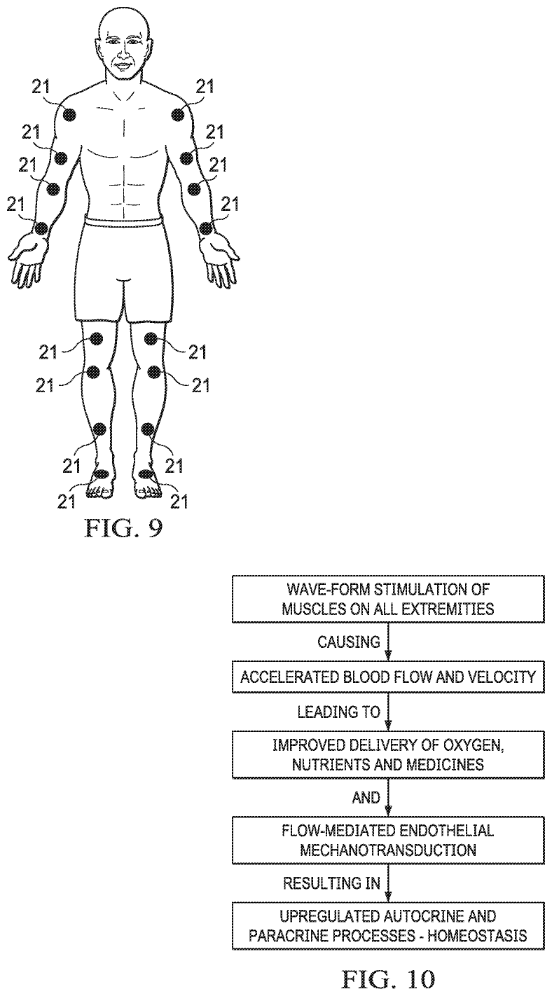

FIG. 10 is a chart of therapeutic results from accelerated wave-form blood movement. Wave-form stimulation of muscles may occur in all extremities, but benefits may also derive from treatment of legs only, for example. Accelerated blood flow and velocity occurs as a result. Improved delivery of oxygen, nutrients, and medicines occur, partially as a result of simply movement of blood. However, methods of the present disclosure also cause flow-mediated endothelial mechanotransduction and its attendant benefits. This results in upregulated autocrine and paracrine processes, for example.

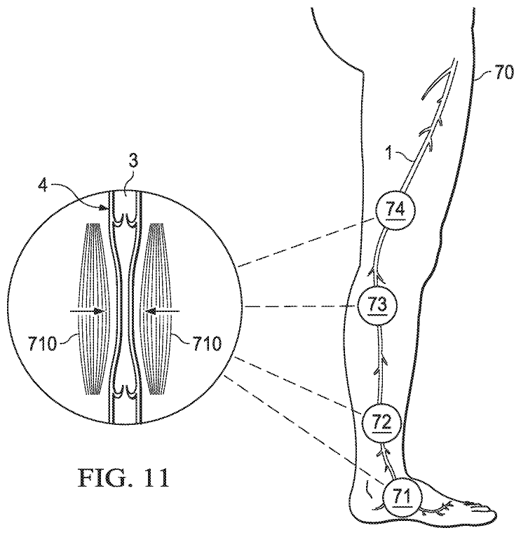

Referring now to FIG. 11 is a simplified diagram of a human patient leg 70 further illustrating possible treatment pad locations is shown. The leg 70 is shown with a large vein 4 running from the foot up to the trunk of the patient. The vein 4 is representative only but could be the great saphenous vein, for example. Location 71 may be a furthest distal location for a pair of pads on or near the foot. Location 72 may be superior to the ankle and more proximal than location 71. Location 73 may be just below the knee, for example, and even more proximal than location 72. Location 74 may be superior to the knee and therefore the most proximal location. The locations 71, 72, 73, 74 provide placements for four pairs of pads capable of executing the sequential, overlapping wave form as discussed elsewhere.

The inset of FIG. 11 shows the anatomical location of skeletal muscle tissue surrounding the blood vessel 4 and contracting under electrical stimulation (e.g., from a pair of opposed treatments pads on the leg). The lumen 3 is thereby squeezed forcing blood away. By sequential activation of the pads as described herein, blood flow can be assured to occur in the proximal direction and back to the heart.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method comprising:
applying a first plurality of pairs of electric treatment pads to a first limb of a patient from a distal to a proximal location on the first limb; and
providing an electrical neuromuscular stimulation to the first plurality of pairs of treatment pads according to a wave-form;
wherein:
the wave-form is applied in a sequential and overlapping manner to the first plurality of pairs of treatment pads such that the electrical neuromuscular stimulation progresses from the distal to the proximal location on the first limb;
the wave-form activates a first most distal pair of pads of the first plurality of treatment pads and thereafter activates a second most distal pair of pads of the first plurality of treatment pads while keeping the first most distal pair of pads of the first plurality of treatment pads activated; and
the wave-form deactivates the first most distal pair of pads of the first plurality of treatment pads when a third most distal pair of pads of the first plurality of treatment pads is activated.

2. The method of claim 1, further comprising:
applying a second plurality of pairs of electric treatment pads to a second limb of the patient from a distal to a proximal location on the second limb; and
providing the electrical neuromuscular stimulation to the second plurality of pairs of treatment pads according to the predetermined wave-form;
wherein:
the wave-form is applied in a sequential and overlapping manner to the second plurality of pairs of treatment pads such that the electrical neuromuscular stimulation progresses from the distal to the proximal location on the second limb;
the wave-form activates a first most distal pair of pads of the second plurality of treatment pads and thereafter activates a second most distal pair of pads of the second plurality of treatment pads while keeping the first most distal pair of pads of the second plurality of treatment pads activated; and
the wave-form deactivates the first most distal pair of pads of the second plurality of treatment pads when a third most distal pair of pads of the second plurality of treatment pads is activated.

3. The method of claim 2, further comprising:
applying a third plurality of pairs of electric treatment pads to a third limb of the patient from a distal to a proximal location on the third limb; and
providing the electrical neuromuscular stimulation to the third plurality of pairs of treatment pads according to the predetermined wave-form;
wherein:
the wave-form is applied in a sequential and overlapping manner to the third plurality of pairs of treatment pads such that the electrical neuromuscular stimulation progresses from the distal to the proximal location on the third limb;
the wave-form activates a first, most distal pair of pads of the third plurality of treatment pads and thereafter activates a second most distal pair of pads of the third plurality of treatment pads while keeping the first most distal pair of pads of the third plurality of treatment pads activated; and
the wave-form deactivates the first most distal pair of pads of the third plurality of treatment pads when a third most distal pair of pads of the third plurality of treatment pads is activated.

4. The method of claim 3, further comprising:
applying a fourth plurality of pairs of electric treatment pads to a fourth limb of the patient from a distal to a proximal location on the fourth limb; and
providing the electrical neuromuscular stimulation to the fourth plurality of pairs of treatment pads according to the predetermined wave-form;
wherein:
the wave-form is applied in a sequential and overlapping manner to the fourth plurality of pairs of treatment pads such that the electrical neuromuscular stimulation progresses from the distal to the proximal location on the fourth limb;
the wave-form activates a first most distal pair of pads of the fourth plurality of treatment pads and thereafter activates a second most distal pair of pads of the fourth plurality of treatment pads while keeping the first most distal pair of pads of the fourth plurality of treatment pads activated; and
the wave-form deactivates the first most distal pair of pads of the fourth plurality of treatment pads when a third most distal pair of pads of the fourth plurality of treatment pads is activated.

5. The method of claim 1, wherein the first limb is an arm.

6. The method of claim 1, wherein the first limb is a leg.

7. The method of claim 2, wherein the first limb and second limb are arms.

8. The method of claim 2, wherein the first limb and second limb are legs.

9. The method of claim 2, wherein the first limb is a leg and the second limb is an arm.

10. The method of claim 4, wherein the first and second limbs are left and right arms, respectively, and the third and fourth limbs are left and right legs, respectively.

11. The method of claim 10, wherein the wave-form is applied to the first and third plurality of treatment pads simultaneously, followed by application of the wave-form to the second and fourth treatment pads simultaneously.

12. The method of claim 11, wherein application of the wave-form to the first and third plurality of treatment pads does not overlap with application of the wave-form to the second and fourth plurality of treatment pads.

13. The method of claim 1, wherein the electrical neuromuscular stimulation at each pair of the first plurality of pairs of treatment pads is about 500 ms in duration.

14. A method comprising:
applying a first plurality of electrically conductive treatment pads along plurality of locations along a first limb of the patient in need of treatment, the locations along the first limb being from distal to proximal; and
providing an electrical wave-form as an application of current to the first plurality of electrically conductive treatment pads;
wherein the electrical wave-form is applied in a sequential and overlapping manner to the first plurality of treatment pads such that an electrical neuromuscular stimulation progresses from the distal to proximal locations on the first limb;
wherein the electrical wave-form applies the electrical neuromuscular stimulation such that stimulation occurs at two adjacent locations on the first limb, except for a beginning of the wave-form when only a most distal location of the first limb receives stimulation and an end of the wave-form when only a most proximal location on the first limb receives stimulation; and
wherein the electrical wave-form maintains stimulation at no more than two adjacent locations on the first limb at any time.

15. The method of claim 14 comprising:
applying a second plurality of electrically conductive treatment pads along plurality of locations along a second limb of a patient in need of treatment, the locations along the first limb being from distal to proximal; and
providing the electrical wave-form as an application of current to the second plurality of electrically conductive treatment pads;
wherein the electrical wave-form is applied in the sequential and overlapping manner to the second plurality of treatment pads such that an electrical neuromuscular stimulation progresses from the distal to proximal locations on the second limb;
wherein the electrical wave-form applies the electrical neuromuscular stimulation such that stimulation occurs at two adjacent locations on the second limb, except for a beginning of the wave-form when only a most distal location of the second limb receives stimulation and an end of the wave-form when only a most proximal location on the second limb receives stimulation; and
wherein the electrical wave-form maintains stimulation at no more than two adjacent locations on the second limb at any time.

16. The method of claim 15, wherein the first limb is an arm of the patient and the second limb is a leg of the patient.

17. The method of claim 16, wherein the electrical wave-form is applied to the plurality of electrically conductive treatment pads on the first limb and to the plurality of electrically conductive treatment pads on the second limb simultaneously.

18. A device comprising:
an electrical wave-form generator;
a first plurality of pairs of electric treatment pads adapted to attach to a first limb of a patient from a distal to a proximal location on the first limb, each of the pairs of electrical treatment pads of the first plurality of pairs of electrical treatment pads, when active, providing electrical neuromuscular stimulation at their respective location to the first limb of the patient; and
leads electrically connecting the first plurality of pairs of electric treatment pads to the wave-form generator;
wherein the electrical wave-form generator provides a stimulation wave-form to the first plurality of pairs of electrical treatment pads;
wherein the stimulation wave-form activates the first plurality of pairs of treatment pads in a sequential and overlapping manner such that the electrical neuromuscular stimulation progresses from the distal to the proximal location on the first limb;
wherein the stimulation wave-form activates a first most distal pair of pads of the first plurality of treatment pads and thereafter activates a second most distal pair of pads of the first plurality of treatment pads while keeping the first most distal pair of pads of the first plurality of treatment pads activated; and
the stimulation wave-form deactivates the first most distal pair of pads of the first plurality of treatment pads when a third most distal pair of pads of the first plurality of treatment pads is activated.

19. The device of claim 18 further comprising:
a second plurality of pairs of electric treatment pads adapted to attach to a second limb of a patient from a distal to a proximal location on the second limb, each of the pairs of electrical treatment pads of the second plurality of pairs of electrical treatment pads, when active, providing electrical neuromuscular stimulation at their respective locations to the second limb of the patient; and
leads electrically connecting the second plurality of pairs of electric treatment pads to the wave-form generator;
wherein the electrical wave-form generator provides the stimulation wave-form to the second plurality of pairs of electrical treatment pads;
wherein the stimulation wave-form activates the second plurality of pairs of treatment pads in a sequential and overlapping manner such that the electrical neuromuscular stimulation progresses from the distal to the proximal location on the second limb;
wherein the stimulation wave-form activates a first most distal pair of pads of the second plurality of treatment pads and thereafter activates a second most distal pair of pads of the second plurality of treatment pads while keeping the first most distal pair of pads of the first plurality of treatment pads activated; and
the stimulation wave-form deactivates the first most distal pair of pads of the second plurality of treatment pads when a third most distal pair of pads of the second plurality of treatment pads is activated.

20. The device of claim 19, wherein the wave-form generator provides the stimulation wave-form to the first and second pluralities of treatment pads simultaneously.
